(19) United States
(12) Patent Application Publication
(10) Pub. No.: US 2025/0189944 A1
(43) Pub. Date: Jun. 12, 2025

(54) ASSISTANCE APPARATUS FOR ISSUING A HANDLING RECOMMENDATION TO AN OPERATOR

(71) Applicant: Siemens Healthineers AG, Forchheim (DE)

(72) Inventors: Jan-Mels Brandt, Herzogenaurach (DE); Sean Harrison, Herzogenaurach (DE)

(21) Appl. No.: 18/972,198

(22) Filed: Dec. 6, 2024

(30) Foreign Application Priority Data
Dec. 6, 2023 (DE) 10 2023 212 265.0

(51) Int. Cl. G05B 19/042

(52) U.S. Cl. CPC G05B 19/042

(57) ABSTRACT

An assistance apparatus for issuing a handling recommendation to an operator who is undertaking an operative intervention on an object includes an object identification unit for identification of the object. The assistance apparatus includes an operator identification unit for identification of the operator, a first memory unit for storage of the characteristic object data describing the identified object, and a second memory unit for storage of the characteristic operator data describing the identified operator. The assistance apparatus includes a specification unit for specifying the intervention to be undertaken on the object, a preparation unit that is in communication with the specification unit and is configured to procure and to store intervention-related operation data. The assistance apparatus includes an evaluation unit configured to evaluate at least the characteristic operator data, the characteristic object data, and the intervention-related operation data, and, depending on the evaluation, establish and issue the handling recommendation.

This application claims the benefit of German Patent Application No. DE 10 2023 212 265.0, filed on Dec. 6, 2023, which is hereby incorporated by reference in its entirety.

### BACKGROUND

The present embodiments relate to an assistance apparatus for issuing a handling recommendation to an operator who is carrying out an operative intervention on an object, a method for issuing a handling recommendation to an operator who is carrying out an operative intervention on an object, and a computer program product.

Assistance apparatuses, methods for assistance apparatuses, and computer program products of the generic kind, and also uses of such methods for assisting an operator during an intervention on an object, are widely known in the prior art. Generic assistance apparatuses, methods, and computer program products serve, for example, to assist an operator who is carrying out an intervention on the object, in order to establish, at least in part, a structure of the object in the region of the intervention and/or to change the structure at least in part. Such interventions are frequently used in materials testing, but are also frequently employed in the area of medical interventions for biological material, living beings (e.g., patients), or the like. The object may accordingly be, for example, an object resulting from an industrial production process, but also an extraction product from mining, a body of a living being (e.g., a patient), or the like.

Nowadays, such interventions are frequently carried out using examination devices, especially imaging devices, such as, for example, magnetic resonance devices, computed tomographs, or the like. This makes it possible to prepare for carrying out the intervention on the object, to monitor the intervention directly, and optimize the intervention, for example, so that a duration of the intervention is as short as possible and/or a reliability of the intervention is as great as possible. At least one region of the object is acquired for this in which the intervention takes place or is to take place. This may lead, however, to the region in which the intervention is to be carried out being poorly accessible, at least in part, for the operator, whereby the intervention overall may be more difficult and time-consuming; above and beyond this, there may also be the danger of intervening on the object in an undesired way in a region in which there is no provision for an intervention. With living beings, this may lead to serious injuries, for example.

In medical engineering, use is made, for example, of magnetic resonance (MR), also referred to as magnetic resonance tomography (MRT) or magnetic resonance imaging (MRI). Here, magnetic field impulses are created by a magnetic field source, and these are applied to the object. The magnetic field may have magnetic flux densities in the range of a few Tesla. As a result of interactions between the object and the magnetic field at an atomic level, MR signals may be triggered in the object, in that, for example, proton spins are aligned. Because of the effect of high-frequency magnetic impulses, the MR signals are created. The MR signals may be detected with a local coil, which may be arranged on the object in the intervention region of the object (e.g., on a surface of the object), and evaluated by an evaluation unit of the magnetic resonance device. Imaging with regard to a structure of the object may be provided as a result of the evaluation, for example.

The magnetic field strengths arising during operation as per specification (e.g., while the examination is being carried out on the object) result in very high values in relation to the magnetic flux density (e.g., in the range of around 0.2 T to around 7 T or the like).

Even if generic assistance apparatuses and methods and also computer program products have been proven, disadvantages remain, however. Previously, the operator has, for example, only been assisted during the intervention on the object to the extent that their actions during the intervention on the object are assisted exclusively from the standpoint of the object. The prior art does not take account of the fact that the operator as an individual may also have an influence on the intervention that the operator is conducting. This may lead, in the prior art, to instructions or recommendations as to actions being issued that cannot be realized by the operator or may only be realized inadequately. The reliability of the intervention and/or also the duration of the intervention on the object may be negatively influenced by this.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, reliability of an intervention on an object or a duration of the intervention on the object may at least be improved.

In relation to an assistance apparatus for issuing a handling recommendation to an operator who is undertaking an operative intervention on an object, it is, for example, provided with the invention that the assistance apparatus has: an object identification unit for identifying the object; an operator identification unit for identifying the operator; a first memory unit for storing the characteristic object data describing the identified project; a second memory unit for storing the characteristic operator data describing the identified operator; a specification unit for specifying the intervention to be carried out on the object; a preparation unit that communicates with the specification unit, which is configured to create and to store intervention-related operation data; and an evaluation unit configured to evaluate at least the characteristic operator data, the characteristic object data, and the intervention-related operation data and, depending on the evaluation, to determine and to issue the handling recommendation.

In relation to a method for issuing a handling recommendation to an operator who is undertaking an operative intervention on an object, the present embodiments provide that: the object is identified by an object identification unit; the operator is identified by an operator identification unit; characteristic object data describing the identified object is stored in a first memory unit; characteristic operator data describing the identified operator is stored in a second memory unit; the intervention to be undertaken on the object is specified by a specification unit; intervention-related operation data is created and stored by a preparation unit that communicates with the specification unit; and at least the characteristic operator data, the characteristic object data, and the intervention-related operation data are evaluated by an evaluation unit, and depending on the evaluation, the handling recommendation is established and issued.

In relation to a computer program product, the present embodiments provide that the computer program product has a program for a processor unit of an assistance apparatus. The program has program code sections for carrying out at least one of the following acts of a method for issuing a handling recommendation to an operator who is undertaking an operative intervention on an object: an object identification unit of the assistance apparatus identifies the object; an operator identification unit of the assistance apparatus identifies the operator; the characteristic object data describing the identified object is stored in a first memory unit of the assistance apparatus; the characteristic operator data describing the identified operator is stored in a second memory unit of the assistance apparatus; a specification unit of the assistance apparatus specifies the intervention to be undertaken on the object; a preparation unit of the assistance apparatus in communication with the specification unit creates and stores intervention-related operation data; an evaluation unit of the assistance apparatus evaluates at least the characteristic operator data, the characteristic object data, and the intervention-related operation data and, depending on the evaluation, establishes and issues the handling recommendation.

The present embodiments are based on the idea that, for example, unlike in the prior art, the assistance apparatus not only takes account of characteristic object data for establishing the handling recommendation, but especially also of characteristic operator data. This makes it possible to establish the handling recommendation for the operator during the intervention significantly more specifically and precisely, in order in this way to improve the reliability of the intervention and/or to shorten the duration of the intervention. What may, for example, be achieved by additionally taking account of the characteristic operator data is that the handling recommendation may be established and issued tailored to the operator. This may avoid the operator receiving a handling recommendation that may only be realized with a great effort or even cannot be realized at all for the operator, or which, for example, is ergonomically unfavorable for the individual operator.

In this context, characteristic operator data may, for example, include physical data of the operator (e.g., data relating to a body size or to dimensions of limbs and/or the like, a level of education of the operator, intervention-related quality data in relation to the operator, and/or the like). Characteristic object data may, for example, be object dimensions, dimensions of limbs of the object, dimensions of organs of the object, further physical object data (e.g., in relation to living beings), an overall physical state, a blood pressure, an oxygen saturation, a body temperature, and/or the like.

The object identification unit makes it possible to identify the object with the aid of characteristics of the object, an identifier of the object, and/or the like. For this purpose, for example, imaging may be used in addition, in which the object is initially acquired by a camera, and a corresponding digitized image is stored. The object identification unit may create a corresponding digital image of the object using a separate camera and compare the digital image with the image already stored in order to identify the object. There is also naturally the option of attaching an identification element to an object (e.g., an identification label, such as an RFID label that has an individual identification) that may be read out wirelessly, for example. The object identification unit may have a corresponding reader unit that is capable of reading the stored identification or the like. Depending on the identified object, the assistance apparatus may access further databases in order, for example, to procure intervention-related characteristic object data. The characteristic object data may, for example, include a geometry, dimensions, and/or further characteristic data of the object.

Correspondingly, the operator identification unit is configured to identify the operator who is carrying out the intervention on the object. The operator identification unit is therefore arranged at least during part of the intervention on the object in the vicinity of the object and may detect the operator for identification (e.g., also while the intervention is being carried out). For this purpose, the operator may, for example, be identified by a camera, but also by entering an identification code in an input unit of the operator identification unit and/or the like. The assistance apparatus, depending on the operator identified, may call up corresponding characteristic operator data from further memory units or databases and make it available. What can be achieved is that, using the operator identification unit, while the intervention is being carried out on the object, the identification of the operator may continue to be monitored. This enables it to be provided that the operator identified is also actually undertaking the intervention. If, for example, there is a change of operator during the intervention, this may be detected using the operator identification unit and taken into account for the further consideration of the handling recommendation.

Using the specification unit, it is possible to specify the intervention to be undertaken on the object. The intervention may, for example, show that there is to be an intervention on the object using an instrument (e.g., the instrument is to be introduced or moved into the object in a region of the intervention to be undertaken). With the instrument, it is possible to intervene at least partly within the object or also on its surface and, for example, to ablate material, to introduce material, to act thermally on the material, and/or the like. The specification unit may, for example, have an input unit, by which it is possible to specify the intervention to be undertaken on the object. An organ to be operated on may be specified, for example, for an operation on a patient. There may, however, also be provision, on a hollow body with a number of chambers, for a specific chamber to be filled with a substance. A plurality of further interventions may be provided.

Intervention-related operation data may be procured and stored by the preparation unit, which communicates with the specification unit. For this, the preparation unit may access further memory units and/or databases in order to be able to obtain intervention-specific data. There may be provision, for example, for the preparation unit, depending on the intervention to be undertaken by the specification unit, to procure specific data for this intervention, which, for example, has already been acquired for other objects for which corresponding interventions have been carried out. Databases relating to an anatomy of the object may also be taken into account here. For this, the assistance apparatus may communicate with the Internet or with a data cloud.

The evaluation unit may have a processor unit and/or use a processor unit of the assistance apparatus at least partly in order to provide the desired functionality. The evaluation unit may, however, also have an electronic hardware circuit at least partly. The evaluation unit is configured to evaluate at least the characteristic operator data, the characteristic object data, and the intervention-related operation data and, depending on the evaluation, to establish and issue the handling recommendation. For this purpose, the evaluation unit may realize corresponding processing of the data, so that the desired handling recommendation may be established. The handling recommendation may be issued, for example, as a kind of signal that may be output by an output unit perceptibly to the operator. The output unit may, for example, be an acoustic, a visual, and/or a haptic output unit. The output unit is not restricted to these embodiments, however. A combination of the aforementioned options may also be provided. The issuing of the handling recommendation may at least also include storage thereof.

The evaluation unit communicates with the further units of the assistance apparatus in order to enable the evaluation unit to realize the corresponding evaluation. The handling recommendation may, for example, be a notification for a specific activity to be carried out by the operator at a specific time. The handling recommendation may also be a notification for the operator not to undertake a specific operator action or activity at this time. For example, the handling recommendation may be a notification to the operator to actuate or to employ one or more treatment instruments that are needed during the intervention in a predetermined way.

It is thus possible with the invention, especially by taking into account the characteristic operator data, to optimize the handling recommendation not only in relation to the object and the intervention to be carried out, but in addition, also while taking into account the characteristics of the operator. It is shown by this that the reliability of carrying out the intervention with a specific identified operator and/or time spent for carrying out the intervention may be optimized or improved.

In accordance with one development, it is provided that the assistance apparatus has a first acquisition unit for acquiring a current situation during the operative intervention on the object and for provision of situation data, where the evaluation unit is configured additionally to evaluate the situation data and additionally to establish the handling recommendation depending on the evaluation of the situation data. This development makes it possible to update the handling recommendation or to output a new or updated handling recommendation if this proves expedient as a result of the current situation data. This provides the opportunity for giving the operator a suitable handling recommendation for a subsequent advance in the operative intervention on the object, so that the operator, while taking into account this handling recommendation, may optimize the intervention carried out by the operator. This development proves advantageous when, during the operative intervention, one or more events occur unexpectedly that may influence the operative intervention or even prevent the operative intervention. There may, for example, be provision for the detection of the current situation at predetermined points in time and/or events to be repeated and for an added or new handling recommendation to be established and issued. This provides the opportunity for monitoring the operative intervention and, for example, at discrete times or continuously, to continually inform the operator about handling recommendations. The acquisition unit may, for example, be an MRT, a CT, an ultrasound measuring device, combinations hereof, or the like. The current situation may reflect current progress of the operative intervention on the object (e.g., the positioning and/or use of an instrument in conjunction with the intervention on the object).

The treatment instrument or the instrument may, for example, be a scalpel, a cannula, a stent, a suture, a needle, combinations hereof, and/or the like. The treatment instrument serves to assist the operative intervention on the object.

The assistance apparatus (e.g., the evaluation unit) may have an electronic circuit and/or a processor unit. A processor unit may, for example, be understood as a data processing device that contains a processing circuit. The processor unit may thus, for example, process data for carrying out computing operations. This may also include operations for carrying out indexed accesses to a data structure (e.g., a Look Up Table (LUT)). The processor unit may, for example, contain one or more computers, one or more microcontrollers, and/or one or more integrated circuits (e.g., one or more Application-Specific Integrated Circuits (ASIC), one or more Field-Programmable Gate Arrays (FPGA), and/or one or more Systems on a Chip (SoC)). The processor unit may also contain one or more processors (e.g., one or more microprocessors, one or more Central Processing Units (CPU), one or more Graphics Processing Units (GPU), and/or one or more signal processors, such as one or more Digital Signal Processors (DSP)). The processor unit may also include a physical or a virtual network of computers or others of the units.

In various example embodiments, the processor unit has one or more hardware and/or software interfaces and/or one or more memory units.

A memory unit in the sense of this disclosure may be configured as a volatile data memory (e.g., as Dynamic Random Access Memory (DRAM) or Static Random Access Memory (SRAM) or as non-volatile data memory, such as Read-Only Memory (ROM), as Programmable Read-Only Memory (PROM), as Erasable Read-Only Memory (EPROM), as Electrically Erasable Read-Only Memory (EEPROM), as flash memory or flash EEPROM, as Ferroelectric Random Access Memory (FRAM), as Magnetoresistive Random Access Memory (MRAM) or as Phase-Change Random Access Memory (PCRAM)).

In accordance with one development, the evaluation unit has a processing unit that uses a machine learning method. The processing unit is trained by an at least two-stage learning method, where, in a first stage, training of the processing unit is carried out at least using the intervention-related operation data. Through the training, in accordance with the first stage, the processing unit is trained in relation to the intended operative intervention. For example, the intervention-related operation data procured and stored by the preparation unit may be included for this purpose. If, for example, an operative intervention on an organ, such as, for example, a liver, of the patient, is intended, available data relating to an operative intervention on the patient from other operations and their courses may be made available in order to train the processing unit accordingly. The processing unit is therefore prepared or set as a result of this training for use for this specific operative intervention. In addition, the characteristic operator data and the characteristic object data may also be included for training. This makes it possible to train the processing unit specifically tailored for the object and the operator.

For this purpose, the evaluation unit (e.g., the processing unit) may have a neural network. Here and below, an artificial neural network may be understood as program code that is stored on a computer-readable memory medium and represents one or more networked artificial neurons or may emulate their function. The program code in this case may also have one or more program code components that, for example, may have different functions. For example, an artificial neural network may implement a non-linear model or a non-linear algorithm that maps the one input to an output, where the input is given by an input feature vector or an input sequence and the output, for example, may include an output category for a classification task, one or more predicted values, or a predicted sequence.

Algorithms for machine learning may be considered as computer algorithms for automatically carrying out a processing task. A processing task may, for example, be understood as a task for extraction of information from available data. For example, the processing task may be executed in a few cases in principle by a human who is capable of understanding information corresponding to the data. In the present context, processing tasks are executed automatically however without human assistance being required.

A computer algorithm may, for example, contain a processing algorithm or an algorithm for data analysis, which is or has been trained by machine learning and, for example, may be based on an artificial neural network (e.g., a convolutional neural network). The computer algorithm may, for example, include an object recognition algorithm, an obstacle recognition algorithm, an object tracking algorithm, a classification algorithm, a semantic segmentation algorithm, and/or a depth estimation algorithm.

Corresponding algorithms may, by analogy, also be executed based on data other than information able to be understood by a human. For example, point clouds or images from infrared cameras, lidar systems, etc. may be evaluated by correspondingly adapted computer algorithms. Strictly speaking, the corresponding algorithms do not involve algorithms for processing, because the corresponding sensors may operate in ranges that are not perceptible for humans (e.g., in the infrared range). Therefore, such algorithms are designated within the framework of the present embodiments for automatic perception. Algorithms for automatic perception thus also include, for example, algorithms for automatic visual perception, but with respect to a human perception, are not restricted to this. Consequently, an algorithm for automatic perception, according to this understanding, may include a computer algorithm for automatic execution of a perception task that is or has been trained by machine learning, for example, and, for example, may be based on an artificial neural network. Such generalized algorithms for automatic perception may also include object detection algorithms, object tracking algorithms, classification algorithms, and/or segmentation algorithms (e.g., semantic segmentation algorithms).

If an artificial neural network is used for implementation of an algorithm for automatic processing, an architecture employed may be that of a convolutional neural network (CNN). For example, a 2D CNN may be applied to corresponding 2D camera images. CNNs may also be used for other algorithms for automatic processing. For example, 3D CNNs, 2D CNNs, or 1D CNNs may be applied to point clouds, depending on the spatial dimensions of the point cloud and the details of processing.

The result or the output of an algorithm for automatic processing may be dependent on the specific underlying processing task. For example, the output of an object recognition algorithm may contain one or more delimitation boxes that define a spatial position and optionally an orientation of one or more corresponding objects in the environment and/or corresponding object classes for the one or more objects. An output of a semantic segmentation algorithm, which is applied to a camera image, may contain a class at pixel level for each pixel of the camera image. Similarly to this, an output of a semantic segmentation algorithm that is applied to a point cloud may contain a corresponding point level class for each of the points. The classes at pixel level or at point level may, for example, define an object type to which the respective pixel or point belongs.

Naturally, the option exists of taking into account further data that may be relevant for the operative intervention. In this way, for example, available supplementary data relating to the object may be taken into account (e.g., data that relates to one or more previous operative interventions on the object, even if these interventions have not taken place in the same region). This also allows account to be taken of object-specific peculiarities during the establishment of the handling recommendation that could be relevant under some circumstances, depending on the course of the operative intervention. This enables the assistance apparatus, the method, and the computer program product to be further improved.

The processing unit may be configured, in a second stage, to carry out training of the processing unit as a kind of adaptive learning at least by using the situation data. For this, the situation data previously occurring during the course of carrying out the operative intervention may be used in order to already train the processing unit additionally during the operative intervention or to carry out adaptive learning based hereon. This allows the evaluation unit (e.g., the processing unit) to already be further trained while the evaluation unit is being used, and, for example, to be further improved during the operative intervention or its adaption.

In one embodiment, the assistance apparatus has an object acquisition unit for acquisition of object characteristics. The object acquisition unit may deliver corresponding object data that may be used additionally by the evaluation unit (e.g., the processing unit). The object characteristics may, for example, feature a temperature, a humidity, a pressure, but also mechanical dimensions, and/or the like. With a dynamic object (e.g., a patient), vital parameters may also be acquired by suitable sensors. Vital parameters for a patient may, for example, be a blood pressure, a pulse frequency, breathing, brain waves, and/or the like. For example, the possibility exists for the body temperature of the object to be acquired by a suitable thermometer (e.g., an infrared thermometer).

In one embodiment, the assistance apparatus has an operator acquisition unit for acquisition of operator characteristics. The operator acquisition unit may have one or more sensors, by which the suitable or desired parameters may be acquired (e.g., in real time). For example, these physical parameters may, for example, be a body size, an arm length, a leg length, a weight, but also vital parameters, such as a heartbeat, a blood pressure, a pulse frequency, a conductivity on the surface of the skin of the operator, and/or the like. This allows corresponding operator characteristics to be acquired and accordingly assigned data to be made available within the assistance apparatus for the function thereof. In one embodiment, this data may additionally be taken into account by the evaluation unit (e.g., the processing unit). The object characteristics may be acquired, for example, by a biomechanical jacket that the operator wears, or the like. Naturally, other or supplementary sensors or sensor units may also be provided, by which the desired parameters of the operator may be acquired. For example, a body temperature of the operator may be acquired by an infrared thermometer.

In one embodiment, the operator acquisition unit is configured to acquire at least a position or an orientation of the operator in relation to an intervention point on the object during the intervention. This enables the handling recommendation additionally to be made as a function of a relative position of the operator in relation to the object. For example, the handling recommendation may include a change of the position of the operator in relation to the object or the like. This makes it possible to guide the operator into a position for carrying out the operative intervention that is as comfortable as possible.

In one embodiment, the assistance apparatus may be configured, while the intervention is being undertaken, to acquire operator-related data. This may, for example, include a strain on the operator in order to relieve the operator with respect to the load on their body. There may be provision for establishing a further load capability of the operator, and, for example, with a long or complicated operative intervention, to suggest to the operator measures for maintaining their performance or for restoring it. These measures may, for example, include taking breaks, having food and/or drinks, and/or the like. The handling instruction may also include a change of operator when the assistance apparatus has established that the operator does not have the required knowledge and/or is not capable of providing the required physical strength for the continuation of the operative intervention.

In one embodiment, the evaluation unit is configured to carry out the second stage of training in addition as a function of the acquired operator-related data. This enables operator-related data obtained during the previous progress of the operative intervention to be included in order to additionally train the evaluation unit (e.g., the processing unit). For example, this enables the establishment and output of the handling recommendation to be adapted so that the operator receives handling instructions tailored to their individual ergonomic area.

In one embodiment, the assistance apparatus is configured, depending on the predefined intervention to be undertaken on the object, to issue at least one handling recommendation before the intervention is undertaken. This makes it possible to supply the operator with corresponding information for preparation of the operative intervention, so that the operator may better plan the operative intervention. Accordingly, there is the possibility, with one or more handling recommendations, to prepare the intervention to be undertaken at least partly (e.g., completely), however (e.g., to simulate it). For example, the handling recommendation may also include a predicted course of the operative intervention, so that the operator may prepare accordingly.

In one embodiment, the assistance apparatus has a device acquisition unit for acquisition of devices for assisting and/or carrying out the intervention in an intervention environment. This makes it possible to take account of the devices when establishing the handling recommendation. For example, the handling recommendation may include the use of a specific device during the operative intervention at a specific time or in a specific situation. The intervention environment relates to an environment in which the operative intervention is carried out on the object. The intervention environment may, for example, be a spatial area that includes the object and the operator as well as, for example, also the assistance apparatus. The intervention environment may, for example, be a room in which the intervention is carried out. The device may, for example, be a treatment instrument or also a sensor unit for acquisition of a respective parameter. The device acquisition unit may, for example, have a camera, by which devices in the intervention environment may be acquired and recognized. The camera may provide corresponding camera data for further use by the assistance apparatus. Using image evaluation, the devices present may be recognized, for example. The possibility also exists of marking one or more devices with a wirelessly identifiable label, such as, for example, an RFID label or the like and to equip the device acquisition unit with a corresponding acquisition unit for acquisition of such identification labels. This enables it to be determined in a simple way which devices are present in the intervention environment and/or how many devices are available in the intervention environment.

In accordance with one development, it is provided that the evaluation unit is configured to establish the handling recommendation additionally depending on the evaluation of the situation data and the devices acquired. This makes it possible to adapt the handling recommendation more specifically to the available boundary conditions, in order in this way to further improve how the operative intervention is carried out. In one embodiment, this data may be used by the processing unit. This data may also serve to additionally train the processing unit adaptively.

The advantages and effects specified for the assistance apparatus of the present embodiments naturally also apply equally for the method of the present embodiments, as well as for the computer program product of the present embodiments, and vice versa. Accordingly, apparatus features may also be formulated as method features and vice versa.

The features and combinations of features given above in the description, as well as the features and combinations of features given below in the figure description and/or shown in the figures alone, are not only able to be used in the combination specified in each case but also in other combinations, without departing from the framework of the invention.

For application cases or application situations that may arise in the method and that are not described here explicitly, there may be provision, in accordance with the method, for an error message and/or a request for entering a user acknowledgement to be output and/or a default setting and/or a predefined initial state to be set.

The features and feature combinations given above in the description as well as also the features and feature combinations given in the description of example embodiments below and/or shown in the figures alone are not only able to be used in the respective combination but also in other combinations. There are thus versions of the present embodiments included or to be seen as disclosed that are not shown and explained explicitly in the figures, but which stem and are able to be created from separate feature combinations from the forms of embodiment explained. The features, functions, and/or effects shown with the aid of the example embodiments may, taken in isolation, each represent individual features, functions, and/or effects of the present embodiments to be considered independently of one another, which each also develop the present embodiments independently of one another. Therefore, example embodiments are also to include combinations other than those in the explained forms of embodiment. What is more, the forms of embodiment described may also be expanded by further of the features, functions, and/or effects of the present embodiments already described.

In the figures, the same reference characters designate the same features or functions.

Independent of the grammatical term usage, individuals with male, female, or other gender identities are included within the term.

### BRIEF DESCRIPTION OF THE DRAWINGS

FIG. 1 shows a magnetic resonance examination arrangement with a magnetic resonance device and a magnetic resonance (MR) local coil connected to the magnetic resonance device, which is arranged on a patient for carrying out an examination on the patient;

FIG. 2 shows a perspective view of the magnetic resonance device in accordance with FIG. 1, with an object table arranged at an edge of a mouth of a through opening of the magnetic resonance device; and

FIG. 3 shows, in a schematic block diagram, a functional execution sequence of the assistance apparatus.

### DETAILED DESCRIPTION

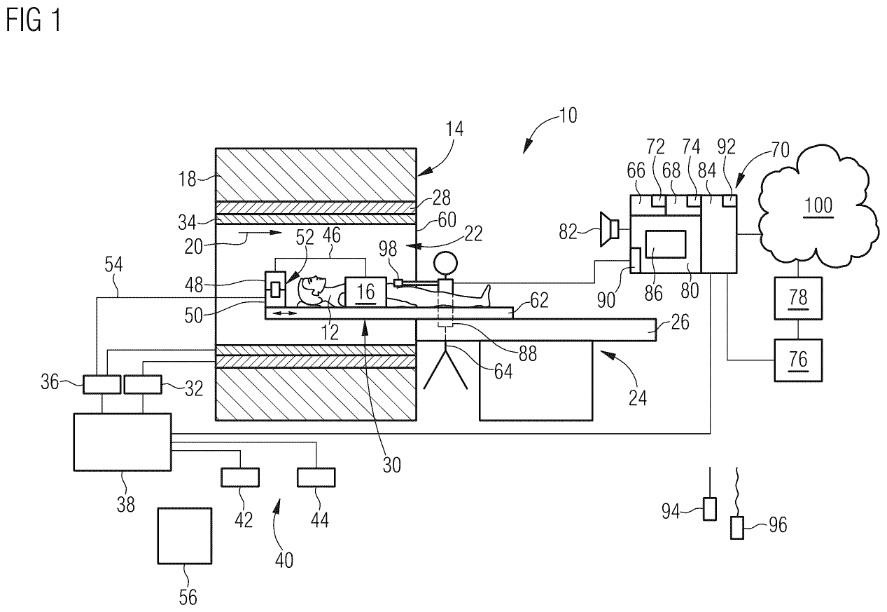

FIG. 1 shows, in a partly sectional diagram, a magnetic resonance examination arrangement 10 that has a magnetic resonance device 14 as an imaging examination device and a local coil 16. The magnetic resonance examination arrangement 10 serves here to carry out an imaging method of a medical examination of a patient 12 as an object to be examined. The examination is carried out during an operative intervention on the patient 12 by an operator 64 in order to monitor the progress of the operative intervention. The operative intervention and the magnetic resonance examination arrangement 10 are, however, not restricted to this application and may be employed in a plurality of other operative interventions on almost any given objects instead of the patient 12.

The magnetic resonance device 14 has a main magnet 18 that serves to create a strong (e.g., temporally constant) main magnetic field in one direction 20. The magnetic resonance device 14 further has a through opening 22 as acquisition area, which, in the present example, is provided as a tunnel and serves to accommodate the patient 12. The through opening 22 in the present example is essentially configured as cylindrical and is surrounded in a circumferential direction by the main magnet 18. Basically, the through opening 22 may, however, also be configured differently from this.

The magnetic resonance device 14 further has a patient support apparatus 24 or an object support apparatus that makes it possible to move the patient 12 or the object into the through opening 22. For this purpose, the patient support apparatus 24 has a patient table 26 arranged movably at least partly within the through opening 22 as the object table. The present embodiments are, however, not restricted to this construction of the acquisition area of the magnetic resonance device 14. There is provision for the examination area 30 in which the operative intervention on the patient 12 is to be carried out, to be arranged in the acquisition area of the magnetic resonance device 14, in order to be able to monitor the course or the progress of the operative intervention.

The magnetic resonance device 14 further has a gradient coil unit 28, by which magnetic field gradients may be created, that may be used for spatial encoding during an imaging examination. The gradient coil unit 28 is controlled in the present example by a gradient control unit 32 of the magnetic resonance device 14. The main magnet 18 and also the gradient coil unit 28 form a magnetic field source.

The magnetic resonance device 14 further has a radio frequency antenna unit 34 that, in the present embodiment, is integrated permanently into the magnetic resonance device 14 as a whole body coil. The radio frequency antenna unit 34 is controlled by a radio frequency antenna control unit 36 of the magnetic resonance device 14. With this, it is possible to create radio frequency magnetic resonance sequences in the examination area 30. In operation of the magnetic resonance device 14, as per specification, an excitation of atomic nuclei in the examination area 30 on the patient 12 may be achieved. By relaxation of the excited atomic nuclei MR, signals are created. The MR signals may be received by the radio frequency antenna unit 34.

For control of the components of the magnetic resonance device 14, the magnetic resonance device 14 has a device control unit 38. The device control unit 38 controls the magnetic resonance examination arrangement 10 centrally (e.g., in relation to carrying out a predetermined examination on the patient 12, such as carrying out a predetermined imaging gradient echo sequence). The device control unit 38 also has an evaluation unit MR not shown in any further detail, by which received MR signals that are acquired during the examination may be evaluated.

The magnetic resonance device 14 further has a user interface 40 that is connected for communication to the control unit 38. The user interface 40 has a display unit 42 and also an input unit 44 that are each connected for communication to the device control unit 38. Via the display unit 42, visual data and information, evaluation results (e.g., imaging results), parameters relating to the MR evaluation, and/or the like may be shown as required. The display unit 42 may, for example, have a monitor or the like. Using the user interface 40, it is possible for a user of the magnetic resonance examination arrangement 10 or the operator 64 to supply data to the device control unit 38 if required (e.g., parameters for carrying out the examination, control data, information, and/or the like).

FIG. 1 shows that a local coil 16 is arranged on the patient 12 in the examination area 30. The local coil 16 is arranged in the present example on a surface of the patient 12 in the examination area 30. The local coil 16 has a connecting cable 46 that is connected in the connection area 52 to a connection cable 54 of the magnetic resonance device 14 for communication and/or for signaling. A coil connection element 48 is provided in the connection area 52 for this purpose, which, in the present example, is permanently connected to the connecting cable 46. The coil connection element 48 is configured, in the present example, as a plug.

A device connection element 50 is further provided in the connection area 52, which, in the present example, is permanently connected to the connection cable 54. With the coil connection element 48 and the device connection element 50, a detachable connection of the local coil 16 to the magnetic resonance device 14 may be realized in the connection area 52. In this way, it is possible to couple a respective local coil 16 to the respective magnetic resonance device 14. The device connection element 50 may be configured as a plug. In the present example, the connection area 52 is a standardized connection area, so that a connection of any given local coils 16 to any given magnetic resonance devices 14 may be achieved based on this standard. Both the coil connection element 48 and also the device connection element 50 have screening in the present example.

In FIG. 1, only a single connection unit 52 for connecting a single local coil 16 is provided. In alternate embodiments, there may naturally be provision for a number of connection units 52 to be provided that allow more than a single local coil to be connected. There may also be provision for the device connection element 50 to be arranged at any given suitable point of the patient table 26. There may also be provision for more than one single device connection element 50 to be provided, where, for example, the number of device connection elements 50 may be arranged at different positions, (e.g., of the patient table 26). The radio frequency antenna unit 34 may, where required, be configured adapted accordingly. This makes it possible to adapt the magnetic resonance examination arrangement 10 as required to the operative intervention to be undertaken.

The magnetic resonance examination arrangement 10 further has an energy supply facility 56. The energy supply facility 56 is connected, in the present example, to a three-phase energy supply network 58.

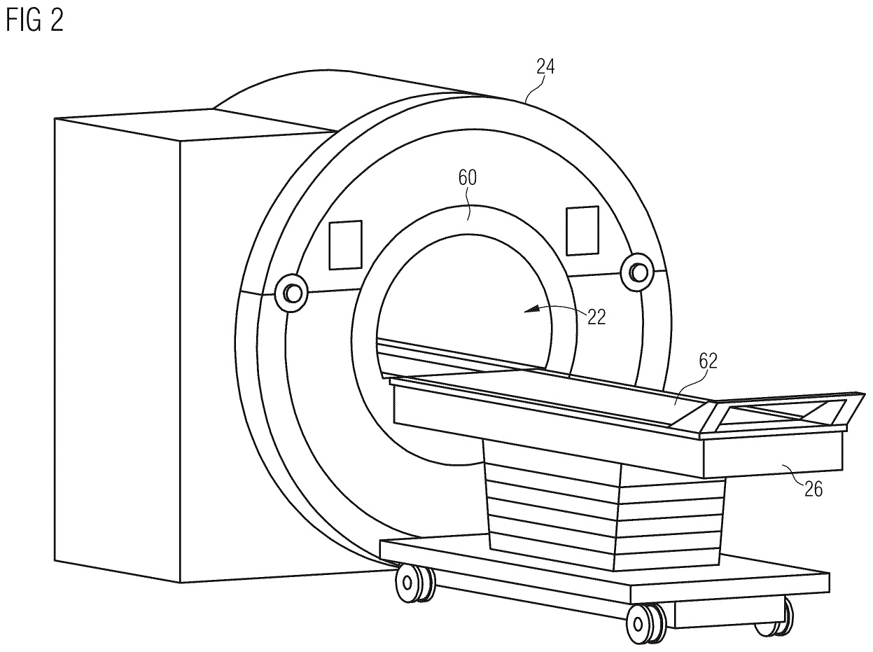

FIG. 2 shows a perspective view of the magnetic resonance device 14 with an object table or patient table 26 arranged at an edge of a mouth 60 of the through opening 22 of the magnetic resonance device 14. The object table or patient table 26 is configured movably in the present example and, for this purpose, has rollers not shown in the figure. The object table 26 may be arranged as required on the magnetic resonance device 14.

The object table or patient table 26 has an object support or patient support 62, on which the patient 12 may be arranged. The patient support 62 is configured to be moved in a longitudinal direction of the patient table 26, so that a patient 12 arranged on the patient support 62 may be arranged as required by the patient support 62 in the through opening 22.

As shown in FIG. 1, an operative intervention is undertaken on the patient 12 who forms the object here. For this purpose, the operator 64 uses a treatment instrument 98 that, in the present example, is formed by a scalpel. For carrying out the operative intervention in the examination area, the scalpel is brought manually by the operator 64 into a region in which the operative intervention is to take place. The operative intervention takes place in the region of the local coil 16, so that the magnetic resonance device 14 is in a position to capture the progress of the operative intervention on the patient 12.

Because of the operation situation and the dimensions of the magnetic resonance device 14, the region at which the operative intervention is carried out on the patient 12 may only be seen with difficulty by the operator 64 and is only accessible in an inconvenient way. Therefore, in order to assist the operator 64 during the operative intervention on the patient 12, an assistance apparatus 70 is provided.

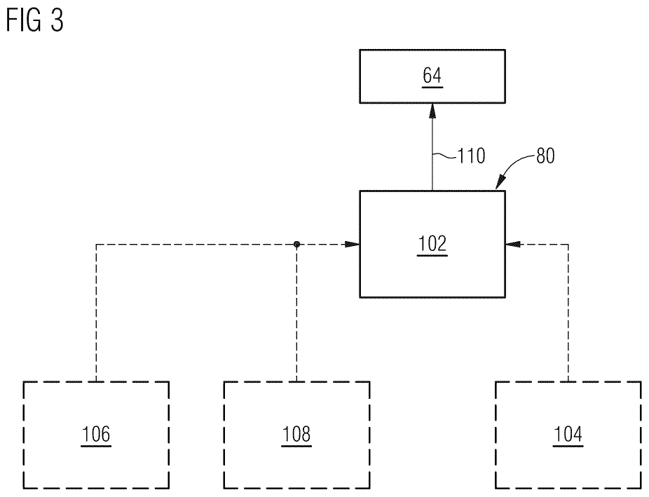

The assistance apparatus 70 is used for issuing a handling recommendation to the operator 64, for which purpose the assistance apparatus 70 has an acoustic output unit in the manner of a loudspeaker 82 that makes it possible to output the handling recommendations 110 (FIG. 3) acoustically to the operator 64. The operator 64 may therefore, using the handling recommendation 110, optimize their activity during the operative intervention on the patient 12 and in this way improve the success of a treatment.

In order to be able to provide the handling recommendations 110, the assistance apparatus 70 has an object identification unit 66 for identification of the object (e.g., the patient 12). The object identification unit 66 may identify the patient 12, for example, using camera images and acquisition of identifying features of the body of the patient 12 and/or the like. What is more an identification is naturally also possible through an identification element, such as, for example, a chip card of the patient 12, which has an individual patient identification, an RFID label that is attached to a part of the body of the patient 12, and/or the like. The object identification unit 66 is configured to detect the chip card or the RFID label and to read out and process the individual identification stored on the chip card or the RFID label.

The assistance apparatus 70 has an operator identification unit 68 for identification of the operator 64. Options corresponding to those for the identification of the patient 12 may be provided, for example, for identification of the operator 64. There may naturally also be provision for the operator 64 to enter an individual personal identification at an input unit of the operator identification unit 68 in order to identify themself to the assistance apparatus 70.

The assistance apparatus 70 further has a first memory unit 72 that serves to store characteristic patient data describing the identified patient 12 as characteristic object data. This may, for example, also be procured by the object identification unit 66 or also by a further unit of the assistance apparatus 70. For this purpose, the assistance apparatus 70 may have a communication connection to further databases that may provide the corresponding characteristic patient data (e.g., body dimensions, a weight of the patient, further medical physiological data such as blood pressure, body temperature, disabilities, and/or the like). The characteristic patient data may also include data that has been acquired in relation to previous operative interventions on the patient 12 (e.g., previous operations), data in relation to an anesthesia in a previous operation, and/or the like.

The assistance apparatus 70 further has a second memory unit 74 that serves to store characteristic operator data describing the identified operator 64. The characteristic operator data may, for example, likewise include body measurements, a level of training, a bodily constitution, and/or the like in relation to the operator 64. This provides that a detailed database in relation to the operator 64 is present in the assistance apparatus 70, so that the handling recommendation 110 may be established and issued individually specifically adapted to characteristics of the operator 64. The operator identification unit 68 or a further unit of the assistance apparatus 70 may further access further databases in order to obtain specific characteristic operator data. These may, for example, also contain data from operative interventions carried out previously.

In the present example, the assistance apparatus 70 may therefore be connected for communication purposes to a data cloud 100 that may include the Internet, for example, and via which it is possible to be able to access a plurality of different databases in order to be able to obtain the desired data.

The assistance apparatus 70 further has a specification unit 76 that serves to specify the intervention to be carried out on the patient 12. For example, there may be provision for the intervention to be undertaken on an organ of the patient 12 (e.g., a liver, a kidney, a bowel, or the like). The specification unit 76 may realize the specification of the operative intervention based on or taking into account characteristic patient data. For this purpose, there may be provision for the specification unit 76 to have an input unit, with which it is made possible for the operator 64 or another member of staff to specify the operative intervention accordingly. The specification unit 76 may also be connected for communication to a remotely arranged input unit, so that the specification of the operative intervention may take place from the remotely arranged location. For example, there may be provision for the specification of the operative intervention on the patient 12 to be specified by a medical specialist who deems the operative intervention to be necessary because of an examination of the patient 12. The operative intervention is then carried by the operator 64 in a hospital, for example. This makes it possible for networking of different institutions (e.g., a hospital and a specialist practice or the like) to be realized and in this way for overall efficiency to be improved.

The assistance apparatus 70 has a preparation unit 78 that is connected for communication to the specification unit 76. The preparation unit 78 serves to prepare for the operative intervention on the patient 12 and to procure and to store intervention-related operation data for this purpose. The intervention-related operation data may also include characteristic patient data. For example, the intervention-related operation data may concern data that relates to the patient 12, and relates, for example, to a region of their body at which the operative intervention is to be undertaken. For example, it is possible for this data to relate to detailed data in relation to an organ of the patient 12 (e.g., a vein route and/or the like).

The preparation unit 78 may use data of the specification unit 76 in order to determine the region on the body of the patient 12 in which the operative intervention is to be undertaken. This may also be used, for example, by the magnetic resonance examination arrangement 10 in order to be able to arrange the patient 12 in the through opening 22 in a suitable way, so that the region in which the operative intervention on the patient 12 is to be carried out may be well acquired by the magnetic resonance device 14.

The assistance apparatus 70 further has an evaluation unit 80 that is configured to evaluate at least the characteristic operator data, the characteristic patient data, and the intervention-related operation data, and depending on the evaluation, to establish the handling recommendation 110. The handling recommendation 110 established in this way may then be output acoustically via the acoustic output unit 82, so that the operator 64 may perceive this acoustically.

In the present example, the evaluation unit 80 has an electronic hardware circuit and a processor unit for this, which realizes the desired functionality using one or more suitable computer program products.

FIG. 3 shows in a schematic flow diagram a functional sequence of the assistance apparatus 70. FIG. 3 shows that the evaluation unit 80 issues to the operator 64 an established handling recommendation 110, as explained above. The evaluation unit 80 has a processing unit 86 that uses a machine learning method. The processing unit 86 is trained by a two-stage learning method, where in a first stage, a training 102 of the processing unit 86 is carried out, at least using the intervention-related operation data 104. In the present example, the training 102 includes, at least based for the present case on an optimized intervention technique, a reduced danger of infection, a reduced stationary stay of the patient 12, and/or the like. The training further includes an improvement of a resection within the framework of the operative intervention, as well as an improvement of the quality of life of the patient 12. The training further includes intervention-specific (e.g., illness-specific) recommendations and summaries, a best operative procedure during the intervention with the fewest complications possible, as well as suggestions for execution and prognosis. For this purpose, the processing unit 86 may have a corresponding processor unit that, for example, provides a neural network that may be trained accordingly by the specified data (e.g., by using data shown or provided in the following acts 104 to 108).

In a first act, there is the training 102 using the data shown or provided in act 104. This involves intervention-related operation data. The data may include registration data (e.g., corresponding data of a community quality initiative, key opinion leader (KOL) data, board data, and/or the like. Stored data that may relate to managing, storage, reporting, to assisting registrations, to providing recommendations of the registration data to work sequences, and the like may be used. Carbon intelligence data may be taken into account, which may involve training, education, and/or colleague data. Further, databases in relation to medical publications, Google Scholar, conference data, and other data may be taken into account. It is further possible to take into account data about support personnel such as carers, nursing staff, anesthetists, radiologists, cardiologists, and/or the like. Finally, as an alternative or in addition, in act 104, medical device data or work sequence data, such as, for example, an angle to be used for a treatment needle, anatomy data (e.g., based on MR data) records, as well as screen data and/or the like may also be taken into account. This data may be procured, at least in part, from other databases via the data cloud 100 or the Internet and stored in the assistance apparatus 70.

While taking account of the aforementioned data, in act 102, there may be corresponding training sequences, which may realize the specified objectives for the act 102.

It is possible to realize a fundamental training of the processing unit 86 additionally while taking account of act 106, in which patient-related data and/or therapy data for the patient 12 is taken into account. In this connection, for example, an EMR, a diagnosis, a patient history, and/or the like may be taken into account.

FIG. 1 shows that the operator 64 is wearing a biometric jacket 88, with which a position, an orientation, and/or a stance of the operator 64 may be acquired. The biometric jacket 88 has a communication connection to an operator acquisition unit 90 of the assistance apparatus 70. Through this (e.g., while using the processing unit 86), a body model of the operator 64 may be created by the evaluation unit 80, on the basis of which the handling recommendations 110 may be established. The body model may be dynamic, which provides that the body model may constantly be adapted as a type of digital twin. For example, this also allows specific operation sections to be simulated during the operative intervention before the operator 64 actually implements the specific operation sections in reality. This may be realized during the operative intervention on the patient 12.

The assistance apparatus 70 further has a first acquisition unit 84 for acquiring a current situation during the operative intervention on the patient 12. The acquisition unit 84 may provide corresponding situation data. The situation data may be made available to the evaluation unit 80 (e.g., to the processing unit 86) in order, through evaluation and supplementary taking into account of the situation data, to establish the handling recommendation. The situation data may, for example, serve, in FIG. 3, in act 108 for training of the processing unit 86 within the framework of a second stage, as a type of adaptive learning. The situation data may, for example, show a relative position of the operator 64 in relation to the patient 12 and/or the like.

Using the evaluation unit 80, it is, for example, possible, even before carrying out the operative intervention, to establish one or more corresponding handling recommendations 110 for carrying out the operative intervention and issue the one or more corresponding handling recommendations 110 to the operator 64. This enables the operator 64 to prepare for the operative intervention in an improved manner.

The assistance apparatus 70 further has a device acquisition unit 92 that is configured to assist devices and/or to acquire the execution of the intervention in an intervention environment. Such devices may, for example, be a treatment needle 94 and an aspiration line 96. The present embodiments are, however, not restricted to this. The devices may also have screens, headphones, tables, the magnetic resonance device 14, an ultrasound device, a C-arm, a CT, navigation systems, and/or the like for registration of a patient, robot facilities and communications facilities, MR data, a wide variety of logs, sequences of operation, as well as also hazards and/or the like.

In the present example, the evaluation unit 80 is further configured to establish the handling recommendation 110 additionally as a function of the evaluation of the situation data and of the acquired devices 94, 96.

The description of the figures serves exclusively to explain the invention and is not intended to restrict the invention.

For example, the invention is naturally not limited to use in the medical field during treatment of a patient. The invention may be employed for almost any given objects, as is also explained in the general description part.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. An assistance apparatus for issuing a handling recommendation to an operator who is undertaking an operative intervention on an object, the assistance apparatus comprising:
an object identification unit configured for identification of the object;
an operator identification unit configured for identification of the operator;
a first memory unit configured to store characteristic object data describing the identified object;
a second memory unit configured to store characteristic operator data describing the identified operator;
a specification unit configured to specify the operative intervention to be undertaken on the object;
a preparation unit that communicates with the specification unit, the preparation unit being configured to procure and to store intervention-related operation data; and
an evaluation unit configured to:
evaluate at least the characteristic operator data, the characteristic object data, and the intervention-related operation data; and
depending on the evaluation, establish and issue the handling recommendation.

2. The assistance apparatus of claim 1, further comprising a first acquisition unit configured to:
acquire a current situation during the operative intervention on the object; and
provide situation data,
wherein the evaluation unit is further configured to:
evaluate the situation data; and
establish the handling recommendation depending on the evaluation of the situation data.

3. The assistance apparatus of claim 1, wherein the evaluation unit comprises:
a processing unit configured to use a machine learning method,
wherein the processing unit is trained by an at least two-stage learning method, and
wherein, in a first stage, a training of the processing unit is carried out at least using the intervention-related operation data.

4. The assistance apparatus of claim 3, wherein the processing unit is further configured, in a second stage, to carry out a training of the processing unit as a kind of adaptive learning, at least while using the situation data.

5. The assistance apparatus of claim 1, further comprising an object acquisition unit configured for acquisition of object characteristics.

6. The assistance apparatus of claim 1, further comprising an operator acquisition unit configured for acquisition of operator characteristics.

7. The assistance apparatus of claim 6, wherein the operator acquisition unit is further configured to acquire at least a position or an orientation of the operator in relation to an intervention point on the object during the intervention.

8. The assistance apparatus of claim 1, wherein the assistance apparatus is configured, while undertaking the intervention, to acquire operator-related data.

9. The assistance apparatus of claim 8, wherein the evaluation unit is further configured to carry out the second stage of the training additionally as a function of the acquired operator-related data.

10. The assistance apparatus of claim 8, wherein the evaluation unit is further configured, during the evaluation, to take account of the acquired operator-related data.

11. The assistance apparatus of claim 1, wherein the assistance apparatus is configured, depending on the specified intervention to be carried out on the object, to issue at least one handling recommendation before the intervention is undertaken.

12. The assistance apparatus of claim 1, further comprising a device acquisition unit configured for acquisition of devices for assisting in, for carrying out, or for assisting in and carrying out the intervention in an intervention environment.

13. The assistance apparatus of claim 11, wherein the evaluation unit is further configured to establish the handling recommendation also depending on the evaluation of the situation data and the acquired devices.

14. A method for issuing a handling recommendation to an operator who is undertaking an operative intervention on an object, the method comprising:
identifying, by an object identification unit, the object;
identifying, by an operator identification unit, the operator;
storing characteristic object data describing the identified object in a first memory unit;
storing characteristic operator data describing the identified operator in a second memory unit;
specifying, by a specification unit, the operative intervention to be undertaken on the object;
procuring and storing, by a preparation unit that communicates with the specification unit, intervention-related operation data;
evaluating, by an evaluation unit, at least the characteristic operator data, the characteristic object data, and the intervention-related operation data; and
depending on the evaluating, establishing and issuing the handling recommendation.

15. In a non-transitory computer-readable storage medium that stores instructions executable by a processor unit of an assistance apparatus for issuing a handling recommendation to an operator who is undertaking an operative intervention on an object, the instructions comprising:
identifying, by an object identification unit of the assistance apparatus, the object;
identifying, by an operator identification unit of the assistance apparatus, the operator;
storing the characteristic object data describing the identified object in a first memory unit of the assistance apparatus;
storing the characteristic operator data describing the identified operator in a second memory unit of the assistance apparatus;
specifying, by a specification unit of the assistance apparatus, the operative intervention to be undertaken on the object;
procuring and storing, by a preparation unit of the assistance apparatus that is in communication with the specification unit, intervention-related operation data;
evaluating, by an evaluation unit of the assistance apparatus, at least the characteristic operator data, the characteristic object data, and the intervention-related operation data; and
depending on the evaluating, establishing and issuing the handling recommendation.
